SEQUENCE LISTING (30 sequences)

SEQ ID NO: 1 - DNA, 51 bp, Homo sapiens
  rs1801020
ataggcagctggaccaacggacggaygccatgagggctctgctgctcctgg

SEQ ID NO: 2 - DNA, 51 bp, Homo sapiens
  rs5985
cctgcccacagtggagcttcagggcdtggtgccccggggcgtcaacctgca

SEQ ID NO: 3 - DNA, 51 bp, Homo sapiens
  rs1799963
gttcccaataaaagtgactctcagcragcctcaatgctcccagtgctattc

SEQ ID NO: 4 - DNA, 51 bp, Homo sapiens
  rs6025
tgtaagagcagatccctggacaggcraggaatacaggtattttgtccttga

SEQ ID NO: 5 - DNA, 50 bp, Homo sapiens
  rs8176719
gcagtaggaaggatgtcctcgtggtaccccttggctggctcccattgtct

SEQ ID NO: 6 - DNA, 51 bp, Homo sapiens
  rs8176719
gcagtaggaaggatgtcctcgtggtgaccccttggctggctcccattgtct

SEQ ID NO: 7 - DNA, 51 bp, Homo sapiens
  rs7853989
acgtcctgccagcgcttgtaggcgcvcacctccagcactgacagctgccga

SEQ ID NO: 8 - DNA, 51 bp, Homo sapiens
  rs8176743
tccgctgttcggcaccctgcaccccrgcttctacggaagcagccgggaggc

SEQ ID NO: 9 - DNA, 50 bp, Homo sapiens
  rs8176750
ccaagaaccaccaggcggtccggaaccgtgagcggctgccaggggctctg

SEQ ID NO: 10 - DNA, 51 bp, Homo sapiens
  rs8176750
ccaagaaccaccaggcggtccggaacccgtgagcggctgccaggggctctg

SEQ ID NO: 11 - DNA, 51 bp, Homo sapiens
  rs2232698
ttcaaacttcggattcagcctgctgygaaagatctccatgaggcacgatgg

SEQ ID NO: 12 - DNA, 51 bp, Homo sapiens
  rs121909548
aaatgaagaaggcagtgaagcagctbcaagtaccgctgttgtgattgctgg

SEQ ID NO: 13 - DNA, 51 bp, Homo sapiens
  rs118203906
attaaaaactgcccaaagaaaaccasgaatcttaagaaaataactcgtgag

SEQ ID NO: 14 - DNA, 51 bp, Homo sapiens
  rs118203905
cattaaaaactgcccaaagaaaaccrggaatcttaagaaaataactcgtga

SEQ ID NO: 15 - DNA, 24 bp, Artificial Sequence
  Primer 9963-F
ttgtgtttctaaaactatggttcc

SEQ ID NO: 16 - DNA, 22 bp, Artificial Sequence
  Primer 9963-R
agtagtattactggctcttcct

SEQ ID NO: 17 - DNA, 22 bp, Artificial Sequence
  Primer 6025-F
tctgaaaggttacttcaaggac

SEQ ID NO: 18 - DNA, 18 bp, Artificial Sequence
  Primer 6025-R
atcgcctctgggctaata

SEQ ID NO: 19 - DNA, 20 bp, Artificial Sequence
  Primer 1020-F
tgatctggactcctggatag

SEQ ID NO: 20 - DNA, 19 bp, Artificial Sequence
  Primer 1020-R
atcctggttcccacagcac

SEQ ID NO: 21 - DNA, 21 bp, Artificial Sequence
  Primer 5985-F
tccacccaataactctaatgc

SEQ ID NO: 22 - DNA, 20 bp, Artificial Sequence
  Primer 5985-F
gtatgctcataccttgcagg

SEQ ID NO: 23 - DNA, 19 bp, Artificial Sequence
  Primer 3989-F
atccacctcgctgaggaag

SEQ ID NO: 24 - DNA, 20 bp, Artificial Sequence
  Primer 3989-R
ccaccgtgtccactactatg

SEQ ID NO: 25 - DNA, 20 bp, Artificial Sequence
  Primer 6719-F
tctccatgtgcagtaggaag

SEQ ID NO: 26 - DNA, 19 bp, Artificial Sequence
  Primer 6719-R
caatggtggtgttctggag

SEQ ID NO: 27 - DNA, 19 bp, Artificial Sequence
  Primer 6743-F
cagcgaggtggattacctg

SEQ ID NO: 28 - DNA, 18 bp, Artificial Sequence
  Primer 6743-R
ccggcgctcgtaggtgaa

SEQ ID NO: 29 - DNA, 19 bp, Artificial Sequence
  Primer 6750-F
gctgaggttcactgcggtg

SEQ ID NO: 30 - DNA, 20 bp, Artificial Sequence
  Primer 6750-R
ttactcacaacaggacggac

(19) United States
(12) Patent Application Publication
(10) Pub. No.: US 2022/0372572 A1
(43) Pub. Date: Nov. 24, 2022

(54) PREDICTION OF PREGNANCY LOSS

(71) Applicant: Genincode UK, Ltd., Manchester (GB)

(72) Inventors: Eduardo Salas, Manchester (GB); José Antonio Páramo, Manchester (GB); Sara Pich, Manchester (GB); Jose Bellver, Manchester (GB); Kevin Guillén, Manchester (GB); Israel Ortega, Manchester (GB); José Manuel Soria, Manchester (GB)

(21) Appl. No.: 16/968,000

(22) PCT Filed: Feb. 8, 2019
(86) PCT No.: PCT/EP2019/053153

§ 371 Date: Aug. 6, 2020

(30) Foreign Application Priority Data
Feb. 9, 2018 (EP) 18382073.7

(51) Int. Cl. C12Q 1/6883; G16B 20/20; G16B 40/20

(52) U.S. Cl. CPC C12Q 1/6883; G16B 20/20; G16B 40/20; C12Q 2600/156; C12Q 2600/118

(57) ABSTRACT

The invention relates to a method for a more appropriate risk assessment for the possible occurrence of a pregnancy loss or recurrent pregnancy loss, based on the presence of different genetic variants. The invention also relates to a method for determining the risk of suffering a a pregnancy loss or RPL by combining the absence or presence several polymorphic markers in a sample from the subject with conventional risk factors as well as computer-implemented means for carrying out said method.

### CROSS REFERENCE TO RELATED APPLICATIONS

This Application is a National stage filing under 35 U.S.C. 371 of International Application No. PCT/EP2019/053153, filed Feb. 8, 2019, which claims priority to European Application No. 18382073.7, filed Feb. 9, 2018, each of which is herein incorporated by reference in their entirety.

### FIELD OF THE INVENTION

The present invention relates to the field of the prediction of a pregnancy loss by screening for thromboembolic diseases or disorders. More specifically, it relates to markers and methods for determining whether a subject, particularly a human female, is at risk of experiencing difficulties with a pregnancy, in particular a pregnancy loss, or even a recurrent pregnancy loss; these methods allow the clinician to potentially prevent such a pregnancy loss by prescribing suitable anti-thrombotic therapy to the female patient at risk.

### TECHNICAL BACKGROUND

Thromboembolic disease is not only the leading cause of morbidity and mortality in the developed world (America Heart Association 2010. Circulation 2010;121:e46-e215), it is also thought be possibly be implicated in pregnancy loss (in the following also “PL”) in human subjects.

Thrombophilia is defined as a hypercoagulable state that leads to thrombotic tendency (Martinelli et al., 2010). Thrombophilia can be inherited, acquired, or mixed (congenital and acquired), and the risk of venous thromboembolism (VTE) differs, based on the resulting modification of the coagulation (Mannucci and Franchini, 2014). Women with thrombophilia are thought to be at increased risk of venous thrombosis during pregnancy, placenta-mediated pregnancy complications, and recurrent pregnancy loss (RPL) (Cao et al., 2013; Ziakas et al., 2015). Although 15% of clinically recognized pregnancies miscarry, the rate of total reproductive losses is closer to 50% (Rai and Regan, 2006). Recurrent pregnancy loss (understood as clinical miscarriages, per the Practice Committee of American Society for Reproductive Medicine, 2013) affects 0.4% to 2% of couples (Cohn et al., 2010)

The most commonly tested types of inherited thrombophilia include deficiencies in antithrombin, protein C, or protein S, but in particular the gain-of-function genetic variants F5-rs6025 and F2-rs1799963 (in the following these two will also be designated as “FVL-PT panel”). Several cohort and case control studies have noted a positive association between thrombophilia and pregnancy loss (Cao et al., 2013; Middeldorp, 2014). The risk for PL is higher in women with inherited thrombophilia; however, the value of thrombophilia screening is unknown even today (Simcox et al., 2015).

The existing approaches for prediction of a possible pregnancy loss or RPL, in particular in the context of thrombophilia, can be summarized as follows:

### Laboratory Testing

Currently, there is no single laboratory global assay that will ‘screen’ for the risk to experience a pregnancy loss or RPL in the context of thrombophilia.

### Specialized Coagulation Testing

Special Coagulation testing consists of a battery of complex (protein and DNA-based) thrombophilia assays to detect presence of an inherited or acquired thrombophilia. However, multiple pre-analytical conditions affect results of the non-DNA-based assays (e.g. anticoagulants, acute thrombosis, liver disease, etc.), so interpretation of results needs to be done within the context of the circumstances surrounding testing.

### Inherited Resistance to Activated Protein C: Factor V Leiden in the Context of Thrombophilia in General

Until 1994, the investigation of patients with clinical evidence of hypercoagulability was usually unproductive, However, with the discovery by Dahlback and Hildebrand of an inherited form of resistance to the proteolytic effects of activated protein C, and the subsequent finding of a common missense mutation in the factor V gene by Bertina and colleagues in Leiden, a major advance was made in the laboratory assessment of thrombotic risk.

The Leiden mutation substitutes a glutamine for an arginine at amino acid residue 506 in factor V, the initial c1 eavage site for activated protein C. The mutation is readily detected by a number of PCR-based approaches. Between 2% and 5% of individuals in Western populations have been documented to be heterozygous for factor V Leiden. In contrast, the mutation is extremely rare in subjects of Asian and African descent,

In some laboratories, initial screening for resistance to activated protein C is performed using the prolongation of an activated partial thromboplastin time-based assay as an indicator; patients testing positive (prolongation in the presence of factor V-deficient plasma) are subsequently evaluated by a PCR.

Increasingly, where access to PCR-based molecular analysis is routine, laboratories will more often choose to proceed directly to the genetic test, as the result is definitive and more than 95% of activated protein C resistance is a result of this single mutation.

Persons heterozygous for the factor V Leiden mutation have an approximately five-fold increased relative risk of venous thrombosis. It is found in 15-20% of patients experiencing their first episode of venous thrombosis and in 50-60% of thrombosis patients with a family history of thrombotic disease. The hypercoagulable phenotype associated with factor V Leiden shows incomplete penetrance, and some individuals may never manifest a clinical thrombotic event. In contrast to the increased relative risk for an initial venous thrombotic event associated with factor V Leiden, this genetic variant is not associated with increased risks for either arterial thrombosis or a recurrence of venous thrombosis. Coinheritance of other inherited thrombotic risk factors or exposure to environmental risk factors can dramatically enhance the thrombotic risk in carriers of factor V Leiden. Many clinicians test for this disorder in patients with a family history of thrombosis who are about to be exposed to an acquired thrombotic risk factor. Individuals homozygous for the mutation have a 70-fold enhanced relative risk of venous thrombosis, indicating that this phenotype is transmitted as a co-dominant trait.

There is no study which conclusively demonstrates the clinical utility of testing the presence of this genetic variant in women with pregnancy loss or RPL.

### D-dimer Blood Testing in the General Context of Thrombophilia

D-dimer is formed when cross-linked fibrin is broken down by plasmin, and levels are usually elevated with e.g. deep vein thrombosis. Normal levels can help to exclude this condition, but elevated D-dimer levels are nonspecific and have low positive predictive value. D-dimer assays differ markedly in their diagnostic properties for thrombosis, A normal result with a very sensitive D-dimer assay (i.e. sensitivity of approximately 98%) excludes thrombosis on its own [i.e. it has a high negative predictive value (NPV)]. However, very sensitive D-dimer tests have a low specificity (approximately 40%), which limits their use because of high false positive rates. In order to exclude thrombosis, a normal result with a less sensitive D-dimer assay (i. e. approximately 85%) needs to be combined with either a low clinical probability or another objective test that has a high NPV. but is non-diagnostic on its own (e.g., negative venous ultrasound of the proximal veins. As less sensitive D-dimer assays are more specific (approximately 70%), they yield fewer false-positive results.

Specificity of D-dimer also decreases with aging and with co-morbid disorders, such as cancer, Consequently, D-dimer testing may have limited value as a diagnostic test for thrombosis.

### Need for New Risk Factors

Despite the above mentioned existence of risk factors and diagnostic tools for early diagnosis of thrombosis in general, there is not a single test or assay or method currently known which would allow the clinician to establish whether or not a female patient would be at risk for a pregnancy loss or an RPL, in relation to thrombosis.

Even among high-risk groups it is not possible to identify individuals who will actually experience a pregnancy loss. In view of the high burden a pregnancy loss places on the patients and their families, its prevention or the precise determination for a possible risk to experience this type of pregnancy loss or even RPL, would be highly desirable.

Several attempts have been done to use molecular diagnostics to identify subjects at high risk to develop a thrombotic and/or thromboembolic event. However, none of these have attempted to particularly provide a risk estimation for pregnancy loss and/or RPL, associated with a thrombotic event.

Accordingly, there is a need for novel markers, including new genetic and/or clinical markers and specific combinations thereof that would successfully and advantageously predict who is at high risk of experiencing a pregnancy loss and/or RPL; preferably, in a way that preventive measures could be implemented to keep that risk at the lowest possible level.

There is also a need for novel markers, including new genetic markers and specific combinations thereof that would successfully and advantageously assist the diagnosis of a risk of a pregnancy loss and/or RPL, preferably in a way that preventive measures could be implemented to keep that risk at the lowest possible level.

### SUMMARY OF THE INVENTION

In a first aspect, the invention provides a method which is suitable to solve the limitations of the methods used nowadays to estimate the risk to experience a pregnancy loss and/or RPL for a particular human female subject.

The method provided according to the present invention solves the limitations of the prior art and achieves the above goal of providing a method which allows to predict the risk of a human female subject, of experiencing a pregnancy loss or miscarriage (used interchangeably in this application), comprising the steps of determining in a sample isolated from said human female subject the presence at least of one of following genetic variants: factor XII C46T (rs1801020), factor XIII Val34Leu (rs5985), Factor II (prothrombin) G20210A (rs1799963), factor V Leiden Arg506GIn (rs6025), ABO-haplotype (consisting of ABO blood group rs8176719, ABO blood group rs7853989, ABO blood group rs8176743, and ABO blood group rs8176750), or respective SNPs in strong linkage disequilibrium with these variants, whereby the presence of these genetic variables (or the absence thereof in the case of any A1 blood group allele) is indicative of a risk of experiencing a miscarriage and/or a recurrent pregnancy loss (RPL); in a preferred embodiment, the above determination is further complemented with the determination of the age of the subject, whereby risk increases with age.

Age has been shown to be the only classical risk factor which actually had a predictive value in the present context.

The above method is clearly superior to the evaluation based on either the earlier know “TiC panel” as well as the standard panel using only the rs6025 and the rs1799963, and actually provides the first known method for such a risk assessment.

To find this solution to the above object the present inventors have set up a study which focused in particular on the determination of preferred risk markers, clinical and genetic, which would be suitable to solve the object.

### STUDY DESIGN, SIZE DURATION

Case-control observational study, with retrospective data analysis, in 180 healthy women with at least one uncomplicated pregnancy to term and no previous miscarriage and 184 cases of idiopathic recurrent pregnancy loss (RPL).

### PARTICIPANTS/MATERIALS, SETTING, METHODS

Two genetic panels were used: the standard FVL-PT panel, which includes F5-rs6025 and F2-rs1799963, and a new thrombophilia-based genetic panel (TiC-RPL) that has been developed in this study, which includes age, F12-rs1801020, F13-rs5985, F2-rs1799963, F5-rs6025, and AB0-rs8176719, rs7853989, rs8176743, and rs8176750. Their predictive ability was assessed in terms of discrimination (AUC), sensitivity, specificity, positive and negative predictive values (PPV, NPV), and positive and negative likelihood ratios (PLR and NLR).

### SUMMARY OF IMPORTANT RESULTS

Globally, TiC-RPL had a better AUC (95% CI) than FVL-PT [0.763 (0.715-0.811) vs 0.540 (0.514-0.567); p<0.0001], with a sensitivity of 70.65%, a specificity of 67.78%, a PPV of 69.15%, an NPV of 69.32%, a PLR of 2.19, and an NLR of 0.43.

In another aspect, the invention relates to methods for the establishing the probability of an individual, namely a human female subject, of presenting a miscarriage and/or RPL based on the presence of one or more of the polymorphisms mentioned above in combination with age as a further risk factor.

In another aspect, the invention relates to methods for the assistance to the diagnosis of an individual, namely a human female subject, of presenting a miscarriage and/or RPL based on the presence of one or more of the polymorphisms mentioned above, preferably in combination with age as a further risk factor.

In another aspect, the invention relates to methods for the establishing the need for preventive measurements to prevent—in an individual, namely a human female subject—a miscarriage and/or RPL based on the presence of one or more of the polymorphisms mentioned above, preferably in combination with age as a further risk factor.

After having determined the risk based on the above combination of risk factors, the clinician can decide whether to administer a drug which is commonly used for prevention of thrombotic events, whereby such drugs are well known in the art.

“Thromboembolic event” in the context of this application should be understood as the alteration of the haemostasis that leads to the development of a blood clot (thrombus) inside a vascular vessel (artery or vein). The thrombus can even obstruct the vascular vessel completely and/or become detached and obstruct another vascular vessel.

“Thromboembolic event” includes among others the following conditions: arterial thrombosis, fatal- and non-fatal myocardial infarction, stroke, transient ischemic attacks, cerebral venous thrombosis, peripheral arteriopathy, deep vein thrombosis and pulmonary embolism.

“Thromboembolic event” in the context of this application is used interchangeably with “thromboembolism”.

“Thromboembolic event” in the context of this application is used interchangeably with “thrombosis”.

“Thromboembolic event” in the context of this application is used interchangeably with “thromboembolic complication”.

“Thrombophilia” in the context of this application should be understood as the disorders of haemostasis that predispose to thrombosis. Included are heritable deficiencies of the natural anticoagulants anti-thrombin, protein C, and protein S and common mutations in the genes encoding clotting factors and acquired thrombophilias such as antiphospholipid antibodies.

The terms “disease” and “disorder” shall be interpreted in the context of this application interchangeably.

The term “miscarriage” or “pregnancy loss” are used interchangeably herein. With the present method, assay and kit, it is possible to predict a lower or higher risk to miscarry, or to even experience multiple miscarriages (used interchangeably with “recurrent pregnancy loss” or “RPL”); it is hypothesized that this increased risk is based on a thrombotic event in the context of, or during, pregnancy.

“Mutation” in the context of this application should be understood as the change of the structure of a gene, resulting in a variant form which may be transmitted to subsequent generations, caused by the alteration of single base units in DNA, or the deletion, insertion, or rearrangement of larger sections of genes or chromosomes.

“Genetic variants” in the context of this application refers to genetic differences both within and among populations. There may be multiple variants of any given gene in the human population (alleles), leading to polymorphism.

The terms “polymorphism” and “single nucleotide polymorphism” (SNP) are used herein interchangeably and relate to a nucleotide sequence variation occurring when a single nucleotide in the genome or another shared sequence differs between members of species or between paired chromosomes in an individual. A SNP can also be designated as a mutation with low allele frequency greater than about 1% in a defined population. Single nucleotide polymorphisms according to the present application may fall within coding sequences of genes, non-coding regions of genes or the intronic regions between genes.

The term “sample”, as used herein, refers to any sample from a biological source and includes, without limitation, cell cultures or extracts thereof, biopsied material obtained from a mammal or extracts thereof, and blood, saliva, urine, feces, semen, tears, or other body fluids or extracts thereof.

In a further aspect, the invention relates to a computer program or a computer-readable media containing means for carrying out any of the methods of the invention.

In yet a further aspect, the invention relates to a kit comprising reagents for detecting the genetic variants factor XII C46T (rs1801020), factor XIII Val34Leu (rs5985), Factor II (prothrombin) G20210A (rs1799963), factor V Leiden Arg506GIn (rs6025), ABO blood group rs8176719, ABO blood group rs7853989, ABO blood group rs8176743, and ABO blood group rs8176750, whereby the kit is to be used for the determination method or diagnostic method as described above and below.

In a preferred embodiment, the kit additionally comprises instructions for use. In a further preferred embodiment, the kit comprises an instruction to further include age as a risk factor for the determination or diagnostic methods as described above and below.

### DETAILED DESCRIPTION OF THE INVENTION

The authors of the present invention have solved the problems identified above in the methods in use nowadays for the calculation of the risk in a subject to develop a miscarriage and/or RPL.

The authors of the present invention have identified a series of genetic variants which are associated with a risk of presenting such a risk, whereby it has been shown that the presently present specific combination is particularly predictive and thus advantageous for the present goal.

The invention is exemplified by the following items:
1. A method for the risk assessment in a human female subject for experiencing a miscarriage and/or recurrent pregnancy loss, comprising the steps of determining in a sample isolated from said subject the presence of factor XII C46T (rs1801020), factor XIII Val34Leu (rs5985), Factor II (prothrombin) G20210A (rs1799963), factor V Leiden Arg506GIn (rs6025), ABO-haplotype (consisting of ABO blood group rs8176719, ABO blood group rs7853989, ABO blood group rs8176743, and ABO blood group rs8176750), or respective SNPs in strong linkage disequilibrium with these variants, whereby the presence of these genetic variables (or the absence thereof in the case of any A1 blood group allele) is indicative of a risk of experiencing a miscarriage and/or a recurrent pregnancy loss (RPL).
2. A method for the diagnosis of a risk for experiencing a miscarriage and/or recurrent pregnancy loss in a human female subject comprising the steps of determining in a sample isolated from said subject the presence of factor XII C46T (rs1801020), factor XIII Val34Leu (rs5985), Factor II (prothrombin) G20210A (rs1799963), factor V Leiden Arg506GIn (rs6025), ABO haplotype (consisting of rs8176719, rs7853989, rs8176743, and rs8176750), or respective SNPs in strong linkage disequilibrium with these variants, whereby the presence of these genetic variables (or the absence thereof in the case of any A1 blood group allele) is indicative of a risk of experiencing a miscarriage and/or a recurrent pregnancy loss (RPL).

Preferably, the variants belonging to the ABO haplotype, which all pertain to one gene, namely the ABO blood group gene, i.e. rs8176719, rs7853989, group rs8176743, and rs8176750 are analysed together as to their haplotype. When the ABO haplotype determines that a subject does not have any A1 blood group allele, i.e. does not belong to the A1 blood group, then this is indicative of increased risk of experiencing a miscarriage and/or a recurrent pregnancy loss.

In other words: the indication “or the absence thereof in the case of any A1 blood group allele” means that if none of the following combinations 1-5 is present, then this would be indicative of a risk of experiencing a miscarriage and/or a recurrent pregnancy loss:

| Combination | Rs8176719 | rs7853989 | rs8176743 | rs8176750 |
|---|---|---|---|---|
| 1 | GG | CC | GG | CC |
| 2 | GG | CC | GG | CdelC |
| 3 | GG | CG | GA | CC |
| 4 | GdelG | CC | GG | CC |
| 5 | GG | CG | GG | CC |

The risk alleles of the indicated genetic variants are as follows:

| rs | Risk allele |
|---|---|
| 6025 | A |
| 5985 | T |
| 1799963 | A |
| 1801020 | T |
| ABO | Non-A1 |

3. A method as defined in any of the items 1 to 2 wherein the method is to determine or diagnose the risk of experiencing an RPL in a subject who has already experienced at least one miscarriage.
4. A method as defined in any of items 1 to 3 further comprising determining the age of the subject.
5. The method according to any one of items 1 to 4 wherein the sample is an oral tissue sample, scraping, or wash or a biological fluid sample, preferably saliva, urine or blood.
6. The method according to any one or more of items 1 to 5 wherein the presence or absence of the polynucleotide is identified by amplifying or failing to amplify an amplification product from the sample, wherein the amplification product is preferably digested with a restriction enzyme before analysis and/or wherein the SNP is identified by hybridizing the nucleic acid sample with a primer label which is a detectable moiety.
7. The method according to any one or more of items 1 to 6 wherein the presence or absence of the polynucleotide is identified by hybridization to specific Hairloop™ probes spotted on a microarray, by allele-specific PCR, by KASP genotyping chemistry or TaqMan Assays.
8. A method of determining the probability of an human female subject of presenting a miscarriage and/or RPL based on the presence of 1 to P classical risk factors and 1 to J polymorphisms selected from the group of factor XII C46T (rs1801020), factor XIII Val34Leu (rs5985), Factor II (prothrombin) G20210A (rs1799963), factor V Leiden Arg506GIn (rs6025), ABO rs8176719, ABO rs7853989, ABO rs8176743, and ABO rs8176750, or respective SNPs in strong linkage disequilibrium with these variants, using the formula:

Estimating the Risk of (Repeated) Pregnancy Loss.

The individual estimation of the risk of (repeated) pregnancy loss is based on a logistic regression model. The aim of this model is to calculate the probability that a person has of presenting (repeated) pregnancy loss according to his/her genetic, sociodemographic and clinical characteristics. To calculate this probability we use the following equation:

Probability (Y=1|X₁, . . . , Xₙ)=1/1+exp(β₀+β₁x₁+ . . . +βₙxₙ₊β_{f·g}x_{f}·x_{g}+ . . . +β_{h·i}xₕ·xᵢ),

Probability (Y=1|X₁, . . . , Xₙ)=1/1+exp(β₀+β1x₁+ . . . +βₙxₙ₊β_{f·g}x_{f}·x_{g}+ . . . +β_{h·i}xₕ·xᵢ),

Probability (Y=1|X₁, . . . , Xₙ)=1/1+exp(β₀+β₁X₁+ . . . +βₙXₙ) Function 1

wherein:
Probability (Y=1|x₁, . . . , xₙ)=probability of presenting a pregnancy loss or a repeated pregnancy loss associated to thrombophilia in a particular individual with concrete and measurable characteristics in a number of variables 1, . . . , n. This probability could range between 0 and 1;
Exp=exponential natural base;
β₀=coefficient that defines the risk (the probability) of a pregnancy loss or a repeated pregnancy loss associated to thrombophilia non related with the variables 1 to n. This coefficient can take a value from −∞ to +∞ and is calculated as the natural

Probability (Y=1|X₁, . . . , Xₙ)=1/1+exp (β₀+β₁x₁+ . . . +βₙxₙ₊β_{f·g}xᵣ·x_{g}+ . . . +β_{h·i}xₕ·xᵢ),

logarithm of the incidence of venous thrombosis
in the population;
β₁=regression coefficient that expresses the risk (higher or lower) to present a pregnancy loss or a repeated pregnancy loss associated to thrombophilia associated with the value/presence of the predictor variable x₁ . This coefficient can take a value from − [symbol] to + [symbol] ;
x₁=value taken by the predictor variable x1 in an individual. The range of possible values depends on the variable;
βₙ=regression coefficient that expresses the risk (higher or lower) to present thrombosis associated with the value/presence of the predictor variable xₙ. This coefficient can take a value from −∞ to +∞;
xₙ=value taken by the predictor variable xₙ in an individual. The range of possible values depends on the variable.
9. The method according to any one or more of the preceding items, wherein no further genetic variables are determined.
10. A computer program or a computer-readable media containing means for carrying out a method as defined in any of items 1 to 9.
11. A kit comprising reagents for detecting the identity of the nucleotide selected from the group of factor XII C46T (rs1801020), factor XIII Val34Leu (rs5985), Factor II (prothrombin) G20210A (rs1799963), factor V Leiden Arg506GIn (rs6025, ABO rs8176719, ABO rs7853989, ABO rs8176743, and ABO rs8176750, or respective SNPs in strong linkage disequilibrium with these variants, and instructions for use.
12. The kit as defined in item 11 which comprises one or more primer pairs specific for the amplification of a region comprising factor XII C46T (rs1801020), factor XIII Val34Leu (rs5985), Factor II (prothrombin) G20210A (rs1799963), factor V Leiden Arg506GIn (rs6025), ABO rs8176719, ABO rs7853989, ABO rs8176743, and ABO rs8176750, or for respective SNPs in strong linkage disequilibrium with these variants.
13. The kit according to item 12, which consists of the primer pairs of item 12, the instructions for use, and reagents suitable for end-point, fluorescence-PCR chemistry.

Real Time PCR is the preferred method to perform amplification and fluorescence measurements. Discrimination is based on the detection of specific signal proportional to the absence/presence of each allele interrogated by the kit.
14. The kit according to item 13, wherein said reagents are dual hydrolysis probes, MgCl₂ and DNA polymerase.
15. The kit according to item 13 or 14, wherein the primer pairs are the following:

| Detected Variant | Primer | Sequence (5′→3′) |
|---|---|---|
| rs1799963 | 9963-F | TTGTGTTTCTAAAACTATGGTTCC |
| | | (SEQ ID NO: 15) |
| | 9963-R | AGTAGTATTACTGGCTCTTCCT |
| | | (SEQ ID NO: 16) |
| rs6025 | 6025-F | TCTGAAAGGTTACTTCAAGGAC |
| | | (SEQ ID NO: 17) |
| | 6025-R | ATCGCCTCTGGGCTAATA |
| | | (SEQ ID NO: 18) |
| rs1801020 | 1020-F | TGATCTGGACTCCTGGATAG |
| | | (SEQ ID NO: 19) |
| | 1020-R | ATCCTGGTTCCCACAGCAC |
| | | (SEQ ID NO: 20) |
| rs5985 | 5985-F | TCCACCCAATAACTCTAATGC |
| | | (SEQ ID NO: 21) |
| | 5985-R | GTATGCTCATACCTTGCAGG |
| | | (SEQ ID NO: 22) |
| rs7853989 | 3989-F | ATCCACCTCGCTGAGGAAG |
| | | (SEQ ID NO: 23) |
| | 3989-R | CCACCGTGTCCACTACTATG |
| | | (SEQ ID NO: 24) |
| rs8176719 | 6719-F | TCTCCATGTGCAGTAGGAAG |
| | | (SEQ ID NO: 25) |
| | 6719-R | CAATGGTGGTGTTCTGGAG |
| | | (SEQ ID NO: 26) |
| rs8176743 | 6743-F | CAGCGAGGTGGATTACCTG |
| | | (SEQ ID NO: 27) |
| | 6743-R | CCGGCGCTCGTAGGTGAA |
| | | (SEQ ID NO: 28) |
| rs8176750 | 6750-F | GCTGAGGTTCACTGCGGTG |
| | | (SEQ ID NO: 29) |
| | 6750-R | TTACTCACAACAGGACGGAC |
| | | (SEQ ID NO: 30) |

The invention is also further described by way of reference to the figures:

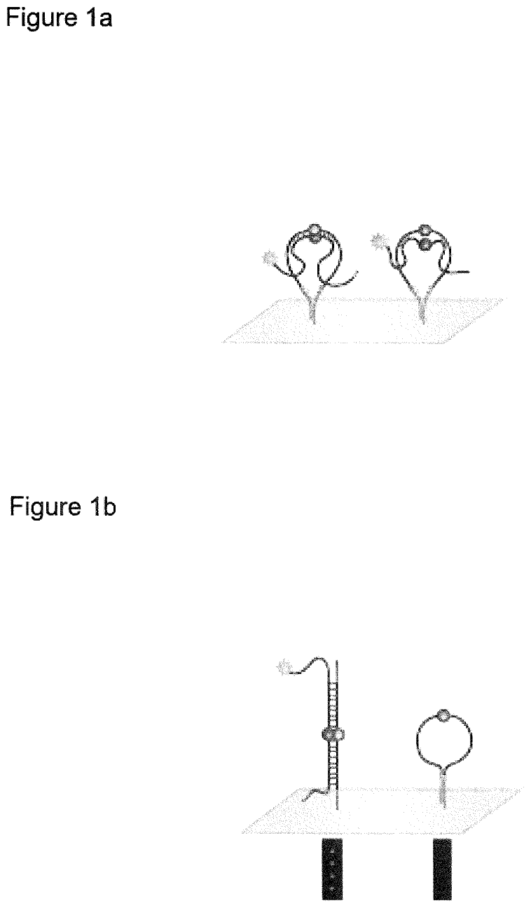

FIG. 1A: HairLoop® molecule, closed state

FIG. 1B: HairLoop® molecule, closed state

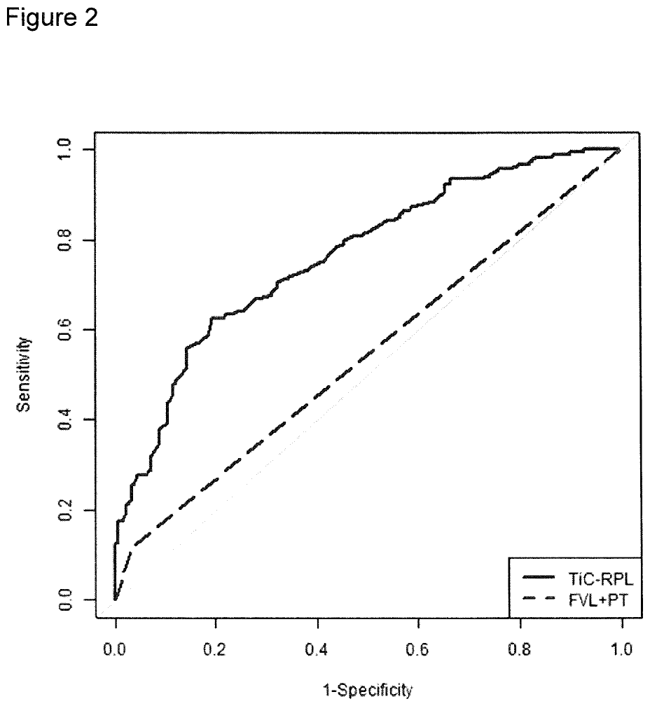

FIG. 2: Area under the ROC curves: FVL-PT and TiC-RPL

A particular combination (as described above) of genetic markers is used, selected and evaluated by the inventors after a complex and genuine analysis of a series of possible markers. Of the different possibilities to construct a genetic risk score (GRS), the inventors have selected the above described particular combination of 8 genetic variants, on the basis of the results as obtained for the different possibilities.

The skilled person may access rs sequences on the NCBI SNP database (“dbSNP”, ncbi.nlm.nih.gov/snp), whereby they are part of the general knowledge of a person skilled in the art.

SNPs in strong linkage disequilibrium can also be used to replace the above specifically recited 8 genetic variants.

Herein, a strong linkage disequilibrium may be defined by the r²value. Linkage disequilibrium is a characterization of the haplotype distribution at a pair of loci. It describes an association between a pair of chromosomal loci in a population. The r² value is considered particularly suitable to describe linkage disequilibrium.

The r² measure of linkage disequilibrium is defined as

$${r}^{2} ( {p}_{a} , {p}_{b} , {p}_{ab} ) = \frac{{( {p}_{ab} - {p}_{a} {p}_{b} )}^{2}}{{p}_{a} ( 1 - {p}_{a} ) {p}_{b} ( 1 - {p}_{b} )} . \qquad ( 1 )$$

where p_{ab} is the frequency of haplotypes having allele a at locus 1 and allele b at locus 2 (Hill & Robertson. 1968). As the square of a correlation coefficient. r²(Pₐ, P_{b}, P_{ab}) can range from 0 to 1 as Pₐ, P_{b}, and P_{ab} vary.

(“Hill & Robertson, 1968” is Theor Appl Genetics 1968;38:226-231).

A strong linkage disequilibrium is one with an r2 value of more than 0.7, preferably more than 0.8, more preferred more than 0.9., including e.g. r² values of 1.

Exemplary for such SNPs in LD are the following:

| | Variant | r2 | D′ | |
|---|---|---|---|---|
| rs6025 | rs9332700 | 1.000 | 1.000 | |
| | rs191160526 | 1.000 | 1.000 | |
| | rs544307093 | 1.000 | 1.000 | |
| | rs548180234 | 1.000 | 1.000 | |
| rs1801020 | rs2545801 | 0.968 | 1.000 | |
| | rs2731672 | 0.938 | 1.000 | |
| | rs2731673 | 1.000 | 1.000 | |
| | rs2731674 | 1.000 | 1.000 | |
| | rs75077631 | 0.968 | 1.000 | |
| rs5985 | rs3024324 | 0.826 | 1.000 | south american population |
| | rs55963140 | 0.922 | 1.000 | south american population |
| | rs3024321 | 0.947 | 1.000 | south american population |
| | rs3024322 | 0.947 | 1.000 | south american population |
| | rs3024323 | 0.947 | 1.000 | south american population |
| | rs3024326 | 0.947 | 1.000 | south american population |
| | rs55742486 | 0.947 | 1.000 | south american population |
| rs1799963 | rs573102333 | 1.000 | 1.000 | south american population |
| rs8176719 | rs8176645 | 0.979 | 1.000 | |
| | rs9411377 | 0.831 | 0.958 | |
| | rs576123 | 0.936 | 0.978 | |
| rs7853989 | rs7855255 | 1.000 | 1.000 | |
| | rs8176751 | 1.000 | 1.000 | |
| | rs8176733 | 1.000 | 1.000 | |
| | rs2073823 | 1.000 | 1.000 | |
| | rs8176730 | 1.000 | 1.000 | |
| | rs8176725 | 1.000 | 1.000 | |
| | rs8176722 | 1.000 | 1.000 | |
| | rs9411365 | 0.801 | 0.927 | |
| | rs9411464 | 0.857 | 1.000 | |
| | rs12216891 | 0.860 | 0.927 | |
| | rs11244049 | 0.860 | 0.927 | |
| | rs8176741 | 0.928 | 1.000 | |
| | rs8176743 | 0.928 | 1.000 | |
| | rs8176746 | 0.928 | 1.000 | |
| | rs8176747 | 0.928 | 1.000 | |
| | rs8176749 | 0.928 | 1.000 | |
| | rs149037075 | 0.928 | 1.000 | |
| | rs187099314 | 0.928 | 1.000 | |
| | rs75179845 | 0.928 | 1.000 | |
| | rs1137827 | 0.928 | 1.000 | |
| | rs8176759 | 0.928 | 1.000 | |
| | rs10793962 | 0.928 | 1.000 | |
| | rs77693339 | 0.928 | 1.000 | |
| | rs10901252 | 0.928 | 1.000 | |
| | rs8176693 | 0.928 | 1.000 | |
| rs8176749 | rs8176747 | 1.000 | 1.000 | |
| | rs8176746 | 1.000 | 1.000 | |
| | rs8176743 | 1.000 | 1.000 | |
| | rs8176741 | 1.000 | 1.000 | |
| | rs149037075 | 1.000 | 1.000 | |
| | rs187099314 | 1.000 | 1.000 | |
| | rs1137827 | 1.000 | 1.000 | |
| | rs8176759 | 1.000 | 1.000 | |
| | rs75179845 | 1.000 | 1.000 | |
| | rs10793962 | 1.000 | 1.000 | |
| | rs77693339 | 1.000 | 1.000 | |
| | rs10901252 | 1.000 | 1.000 | |
| | rs8176693 | 1.000 | 1.000 | |
| | rs11244038 | 0.848 | 1.000 | |
| | rs7467847 | 0.851 | 0.923 | |
| | rs7470777 | 0.851 | 0.923 | |
| | rs9411365 | 0.865 | 1.000 | |
| | rs9411464 | 0.924 | 1.000 | |
| | rs8176751 | 0.928 | 1.000 | |
| | rs7853989 | 0.928 | 1.000 | |
| | rs7855255 | 0.928 | 1.000 | |
| | rs8176733 | 0.928 | 1.000 | |
| | rs2073823 | 0.928 | 1.000 | |
| | rs8176730 | 0.928 | 1.000 | |
| | rs8176725 | 0.928 | 1.000 | |
| | rs8176722 | 0.928 | 1.000 | |
| | rs12216891 | 0.928 | 1.000 | |
| | rs11244049 | 0.928 | 1.000 | |
| rs8176750 | — | — | — | |

When prediction models are used, as for instance, for making treatment decisions regarding the possibility of a treatment with anti-thrombotic and/or anticoagulant drugs, such as but not limited to low molecular weight heparin, aspirin, unfructionated heparin, fondaparinux, bivalirrudin, ximelagatran, warfarin, diphenadion, ximelagatran,dazoxiben, sulphinpyrazone, epoprostenol, dipyridamole, pentoxiphylline, ticlopidine, clopidogrel, abciximab, tirofiban, integrelin, eptifibative, predictive risks may be categorized by using risk cutoff thresholds.

Those skilled in the art will readily recognize that the analysis of the nucleotides present according to the method of the invention in an individual's nucleic acid can be done by any method or technique capable of determining nucleotides present in a polymorphic site. As it is obvious in the art, the nucleotides present in the polymorphic markers can be determined from either nucleic acid strand or from both strands.

Once a biological sample from a subject has been obtained (e.g., a bodily fluid, such as urine, saliva, plasma, serum, or a tissue sample, such as a buccal tissue sample or a buccal cell) detection of a sequence variation or allelic variant SNP is typically undertaken. Virtually any method known to the skilled artisan can be employed. Perhaps the most direct method is to actually determine the sequence of either genomic DNA or cDNA and compare these sequences to the known alleles SNPs of the gene. This can be a fairly expensive and time-consuming process. Nevertheless, this technology is quite common and is well known.

Any of a variety of methods that exist for detecting sequence variations may be used in the methods of the invention. The particular method used is not important in the estimation of cardiovascular risk or treatment selection.

Other possible commercially available methods exist for the high throughput SNP identification not using direct sequencing technologies, for example, Illumina's Veracode Technology, allele-specific PCT with Dynamic Array IFCs, Taqman® SNP Genotyping Chemistry and KASPar SNP genotyping Chemistry.

A variation on the direct sequence determination method is the Gene Chip™ method available from Affymetrix. Alternatively, robust and less expensive ways of detecting DNA sequence variation are also commercially available.

For example, Perkin Elmer adapted its TAQman Assay™ to detect sequence variation. Orchid BioSciences has a method called SNP-IT™ (SNP-Identification Technology) that uses primer extension with labelled nucleotide analogues to determine which nucleotide occurs at the position immediately 3′ of an oligonucleotide probe, the extended base is then identified using direct fluorescence, an indirect colorimetric assay, mass spectrometry, or fluorescence polarization. Sequenom uses a hybridization capture technology plus MALDI-TOF (Matrix Assisted Laser Desorption/lonization-Time-of-Flight mass spectrometry) to detect SNP genotypes with their MassARRAY™ system. Promega provides the READIT™ SNP/Genotyping System (U.S. Pat. No. 6,159,693). In this method, DNA or RNA probes are hybridized to target nucleic acid sequences. Probes that are complementary to the target sequence at each base are depolymerized with a proprietary mixture of enzymes, while probes which differ from the target at the interrogation position remain intact. The method uses pyro-phosphorylation chemistry in combination with luciferase detection to provide a highly sensitive and adaptable SNP scoring system. Third Wave Technologies has the Invader OS™ method that uses proprietary Cleavaseg enzymes, which recognize and cut only the specific structure formed during the Invader process. Invader OS relies on linear amplification of the signal generated by the Invader process, rather than on exponential amplification of the target. The Invader OS assay does not utilize PCR in any part of the assay. In addition, there are a number of forensic DNA testing labs and many research labs that use gene-specific PCR, followed by restriction endonuclease digestion and gel electrophoresis (or other size separation technology) to detect restriction fragment length polymorphisms (RFLPs).

In various embodiments of any of the above aspects, the presence or absence of the SNPs is identified by amplifying or failing to amplify an amplification product from the sample. Polynucleotide amplifications are typically template-dependent. Such amplifications generally rely on the existence of a template strand to make additional copies of the template. Primers are short nucleic acids that are capable of priming the synthesis of a nascent nucleic acid in a template-dependent process, which hybridize to the template strand. Typically, primers are from ten to thirty base pairs in length, but longer sequences can be employed. Primers may be provided in double-stranded and/or single-stranded form, although the single-stranded form generally is preferred. Often, pairs of primers are designed to selectively hybridize to distinct regions of a template nucleic acid, and are contacted with the template DNA under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids containing one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

### Polymerase Chain Reaction

A number of template dependent processes are available to amplify the oligonucleotide sequences present in a given template sample. One of the best known amplification methods is the polymerase chain reaction. In PCR, pairs of primers that selectively hybridize to nucleic acids are used under conditions that permit selective hybridization. The term “primer”, as used herein, encompasses any nucleic acid that is capable of priming the synthesis of a nascent nucleic acid in a template-dependent process. Primers may be provided in double-stranded or single-stranded form, although the single-stranded form is preferred. Primers are used in any one of a number of template dependent processes to amplify the target gene sequences present in a given template sample. One of the best known amplification methods is PCR, which is described in detail in U.S. Pat. Nos. 4,683,195, 4,683,202 and 4,800,159, each incorporated herein by reference. In PCR, two primer sequences are prepared which are complementary to regions on opposite complementary strands of the target-gene(s) sequence. The primers will hybridize to form a nucleic-acid:primer complex if the target-gene(s) sequence is present in a sample. An excess of deoxyribonucleoside triphosphates is added to a reaction mixture along with a DNA polymerase, e.g. Taq polymerase, that facilitates template-dependent nucleic acid synthesis. If the target-gene(s) sequence:primer complex has been formed, the polymerase will cause the primers to be extended along the target-gene(s) sequence by adding on nucleotides. By raising and lowering the temperature of the reaction mixture, the extended primers will dissociate from the target-gene(s) to form reaction products, excess primers will bind to the target-gene(s) and to the reaction products and the process is repeated. These multiple rounds of amplification, referred to as “cycles”, are conducted until a sufficient amount of amplification product is produced.

The amplification product may be digested with a restriction enzyme before analysis. In still other embodiments of any of the above aspects, the presence or absence of the SNP is identified by hybridizing the nucleic acid sample with a primer labelled with a detectable moiety. In other embodiments of any of the above aspects, the detectable moiety is detected in an enzymatic assay, immunoassay, or by detecting fluorescence. In other embodiments of any of the above aspects, the primer is labelled with a detectable dye (e.g., SYBR Green I, YO-PRO-I, thiazole orange, Hex, pico green, edans, fluorescein, FAM, or TET). In other embodiments of any of the above aspects, the primers are located on a chip. In other embodiments of any of the above aspects, the primers for amplification are specific for said SNPs.

Another method for amplification is the ligase chain reaction (“LCR”). LCR differs from PCR because it amplifies the probe molecule rather than producing an amplicon through polymerization of nucleotides. In LCR, two complementary probe pairs are prepared, and in the presence of a target sequence, each pair will bind to opposite complementary strands of the target such that they abut. In the presence of a ligase, the two probe pairs will link to form a single unit. By temperature cycling, as in PCR, bound ligated units dissociate from the target and then serve as “target sequences” for ligation of excess probe pairs. U.S. Pat. No. 4,883,750, incorporated herein by reference, describes a method similar to LCR for binding probe pairs to a target sequence.

### Isothermal Amplification

An isothermal amplification method, in which restriction endonucleases and ligases are used to achieve the amplification of target molecules that contain nucleotide 5′-[[alpha]-thio]-triphosphates in one strand of a restriction site also may be useful in the amplification of nucleic acids in the present invention. In one embodiment, loop-mediated isothermal amplification (LAMP) method is used for single nucleotide polymorphism (SNP) typing.

### Strand Displacement Amplification

Strand Displacement Amplification (SDA) is another method of carrying out isothermal amplification of nucleic acids which involves multiple rounds of strand displacement and synthesis, i.e., nick translation. A similar method, called Repair Chain Reaction (RCR), involves annealing several probes throughout a region targeted for amplification, followed by a repair reaction in which only two of the four bases are present. The other two bases can be added as biotinylated derivatives for easy detection.

Other amplification methods may be used in accordance with the present invention. In one embodiment, “modified” primers are used in a PCR-like, template and enzyme dependent synthesis. The primers may be modified by labelling with a capture moiety (e.g., biotin) and/or a detector moiety (e.g., enzyme). In the presence of a target sequence, the probe binds and is cleaved catalytically. After cleavage, the target sequence is released intact to be bound by excess probe. Cleavage of the labelled probe signals the presence of the target sequence. In another approach, a nucleic acid amplification process involves cyclically synthesizing single-stranded RNA (“ssRNA”), ssDNA, and double-stranded DNA (dsDNA), which may be used in accordance with the present invention. The ssRNA is a first template for a first primer oligonucleotide, which is elongated by reverse transcriptase (RNA-dependent DNA polymerase). The RNA is then removed from the resulting DNA:RNA duplex by the action of ribonuclease H (RNase H, an RNase specific for RNA in duplex with either DNA or RNA). The resultant ssDNA is a second template for a second primer, which also includes the sequences of an RNA polymerase promoter (exemplified by T7 RNA polymerase) 5′ to its homology to the template. This primer is then extended by DNA polymerase (exemplified by the large “Klenow” fragment of E. coli DNA polymerase I), resulting in a double-stranded DNA (“dsDNA”) molecule, having a sequence identical to that of the original RNA between the primers and having additionally, at one end, a promoter sequence. This promoter sequence can be used by the appropriate RNA polymerase to make many RNA copies of the DNA. These copies can then re-enter the cycle leading to very swift amplification. With proper choice of enzymes, this amplification can be done isothermally without addition of enzymes at each cycle. Because of the cyclical nature of this process, the starting sequence can be chosen to be in the form of either DNA or RNA.

It is also conceivable to use massive sequencing, i.e. massive parallel sequencing or massively parallel sequencing, which is any of several high-throughput approaches to DNA sequencing using the concept of massively parallel processing; it is also called next-generation sequencing (NGS). Some of these technologies emerged in 1994-1998 and have been commercially available since 2005. These technologies use miniaturized and parallelized platforms for sequencing of 1 million to 43 billion short reads (50-400 bases each) per instrument run.

It is furthermore conceivable to use the exome as basis for the analysis; The exome is the part of the genome formed by exons, the sequences which when transcribed remain within the mature RNA after introns are removed by RNA splicing. It consists of all DNA that is transcribed into mature RNA in cells of any type as distinct from the transcriptome, which is the RNA that has been transcribed only in a specific cell population. The exome of the human genome consists of roughly 180,000 exons constituting about 1% of the total genome, or about 30 megabases of DNA. Though comprising a very small fraction of the genome, mutations in the exome are thought to harbor 85% of mutations that have a large effect on disease. Exome sequencing has proved to be an efficient strategy to determine the genetic basis of more than two dozen Mendelian or single gene disorders. Regularly, exome sequencing is generated by means of massively parallel sequencing as described before.

### Methods for Nucleic Acid Separation

It may be desirable to separate nucleic acid products from other materials, such as template and excess primer. In one embodiment, amplification products are separated by agarose, agarose-acrylamide or polyacrylamide gel electrophoresis using standard methods (Sambrook et al., 1989, see infra). Separated amplification products may be cut out and eluted from the gel for further manipulation. Using low melting point agarose gels, the separated band may be removed by heating the gel, followed by extraction of the nucleic acid. Separation of nucleic acids may also be effected by chromatographic techniques known in the art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC. In certain embodiments, the amplification products are visualized. A typical visualization method involves staining of a gel with ethidium bromide and visualization of bands under UV light. Alternatively, if the amplification products are integrally labeled with radio- or fluorometrically-labeled nucleotides, the separated amplification products can be exposed to X-ray film or visualized with light exhibiting the appropriate excitatory spectra.

Nucleic acid molecules useful for hybridisation in the methods of the invention include any nucleic acid molecule which exhibits substantial identity so as to be able to specifically hybridise with the target nucleic acids. Polynucleotides having “substantial identity” to an endogenous sequence are typically capable of hybridizing with at least one strand of a double-stranded nucleic acid molecule. By “substantially identical” is meant a polypeptide or nucleic acid molecule exhibiting at least 50% identity to a reference amino acid sequence or nucleic acid sequence. Preferably, such a sequence is at least 60%, more preferably 80% or 85%, and more preferably 90%, 95% or even 99% identical at the amino acid level or nucleic acid to the sequence used for comparison. Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e<″3>and e<″100>indicating a closely related sequence.

A detection system may be used to measure the absence, presence, and amount of hybridization for all of the distinct sequences simultaneously. Preferably, a scanner is used to determine the levels and patterns of fluorescence.

Another method for detecting sequence variations is based on the amplification by PCR of specific human targets and the subsequent detection of their genotype by hybridization to specific Hairloop™ probes spotted on a microarray.

HairLoop™ is a stem-loop, single-stranded DNA molecule consisting of a probe sequence embedded between complementary sequences that form a hairpin stem. The stem is attached to the microarray surface by only one of its strands. In the absence of a DNA target, the HairLoop™ is held in the closed state (FIG. 1a). When the target binds perfectly (no mismatch) to its HairLoop™, the greater stability of the probe-target duplex forces the stem to unwind, resulting in an opening of the HairLoop™ (FIG. 1b). Due to these unique structural and thermodynamic properties, HairLoop™ offer several advantages over linear probes, one of which is their increased specificity differentiating between two DNA target sequences that differ by as little as a single nucleotide.

HairLoop™ act like switches that are normally closed, or “off”. Binding to fluorescent DNA target induces conformational changes that open the structure and as a result after washing, the fluorescence is visible, or “on”.

One HairLoop™ is designed to be specific to one given allele. Thus, assessment of a point mutation for a bi-allelic marker requires two HairLoop™; one for the wild-type allele, and one for the mutant allele.

Surprisingly, the combination of SNP markers included in the present invention and set forth above have proved to be capable to assist in the determination and diagnostic methods of a miscarriage and/or RPL in a human female subject.

In a preferred embodiment, age is included in the risk determination as a further risk marker.

In a further preferred embodiment, the risk is determined with the following function:

Estimating the Risk of (Repeated) Pregnancy Loss.

The individual estimation of the risk of (repeated) pregnancy loss is based on a logistic regression model. The aim of this model is to calculate the probability that a person has of presenting (repeated) pregnancy loss according to his/her genetic, sociodemographic and clinical characteristics. To calculate this probability we use the following equation:

Probability (Y=1|X₁, . . . , Xₙ)=1/1+exp(β₀+β₁x₁+ . . . +βₙxₙ₊β_{f·g}x_{f}·x_{g}+ . . . +β_{h·i}xₕ·xᵢ),

Probability (Y=1|X₁, . . . , Xₙ)=1/1+exp(β₀+β₁x₁+ . . . +βₙxₙ₊β_{f·g}x_{f}·x_{g}+ . . . +β_{h·i}xₕ·xᵢ),

Probability (Y=1|X₁, . . . , Xₙ)=1/1+exp(β₀+β₁X₁+ . . . +βₙXₙ) Function 1

wherein:
Probability (Y=1|x₁, . . . , xₙ)=probability of presenting a pregnancy loss or a repeated pregnancy loss associated to thrombophilia in a particular individual with concrete and measurable characteristics in a number of variables 1, . . . , n. This probability could range between 0 and 1;
Exp=exponential natural base;

Probability (Y=1|X₁, . . . , Xₙ)=1/1+exp(β₀+β₁x₁+ . . . +βₙxₙ₊β_{f·g}xᵣ·x_{g}+ . . . +β_{h·i}xₕ·xᵢ),
β₀=coefficient that defines the risk (the probability) of a pregnancy loss or a repeated pregnancy loss associated to thrombophilia non related with the variables 1 to n. This coefficient can take a value from −∞ to +∞ and is calculated as the natural logarithm of the incidence of venous thrombosis in the population;
β₁=regression coefficient that expresses the risk (higher or lower) to present a pregnancy loss or a repeated pregnancy loss associated to thrombophilia associated with the value/presence of the predictor variable x₁. This coefficient can take a value from − [symbol] to + [symbol] ;
x₁=value taken by the predictor variable x1 in an individual. The range of possible values depends on the variable;
βₙ=regression coefficient that expresses the risk (higher or lower) to present thrombosis associated with the value/presence of the predictor variable xₙ. This coefficient can take a value from −∞ to +∞;
xₙ=value taken by the predictor variable xₙ in an individual. The range of possible values depends on the variable.

The variables included in the model and the regression coefficients of each of these variables are shown in the following table 1.

TABLE 1

| | | | Regression | Regresion |
|---|---|---|---|---|
| | | Regression | coefficient | coeficient |
| Variable | Risk Exposure | Coefficient | lower limit | upper limit |
| Age | Yes | 0.2159 | 0.01 | 3 |
| Factor V Leiden Heterozygote | AG | 0.6981347 | 0.01 | 3 |
| Factor V Leiden Homozygote | AA | 1.3963 | 0.01 | 3 |
| F2 Prothrombin Heterozygote | AG | 0.415672 | 0.01 | 3 |
| F2 Prothrombin Homozygote | AA | 1.6831 | 0.01 | 3 |
| Factor 12 Heterozygote | CT | 0.5108 | 0.01 | 3 |
| Factor 12 Homozygote | TT | 1.0216 | 0.01 | 3 |
| Factor 13 Heterozygote | GT | 0.3999 | 0.01 | 3 |
| Factor 13 Homozygote | TT | 0.79998 | 0.01 | 3 |
| ABO (none A1 allele) | (see next table) | 0.367 | 0.01 | 3 |

TABLE 2
None of the following combination (A1 allele)

| Combination | Rs8176719 | rs7853989 | rs8176743 | rs8176750 |
|---|---|---|---|---|
| 1 | GG | CC | GG | CC |
| 2 | GG | CC | GG | CdelC |
| 3 | GG | CG | GA | CC |
| 4 | GdelG | CC | GG | CC |
| 5 | GG | CG | GG | CC |

By the use of the methods and functions described, a personalized risk is obtained for experiencing a miscarriage or RPL.

The inventors diligently conducted a study to achieve their goal of providing an improved method for the risk estimation of a miscarriage or RPL, as shown in the following example:

### EXAMPLE

### Materials and Methods

Ethical Approval

This study was registered at ClinicalTrials.gov under registry number NCT03336463. The study was conducted in compliance with the Helsinki Declaration and was approved by the corresponding institutional ethics committees. All patients signed informed consent forms before inclusion.

Study Design and Participants

This multi-center, case-control, observational study, with retrospective data analysis, was performed in 4 centers throughout Spain, from three geographical areas.

RPL women were eligible for study participation if they fulfilled the following criteria: age >18 years, a history of RPL (≥2 consecutive or ≥3 non-consecutive) from spontaneous or assisted pregnancies, use of their own gametes, normal karyotype in both members of the couple, normal or corrected thyroid function, BMI <30, normal uterine anatomy (as assessed by 3D ultrasound, hysterosalpingography, or hysteroscopy), nondiabetic, no chronic pathologies, no hydrosalpinx, and not taking concomitant anticoagulant or anti-aggregant therapies. The couple's sperm could be analyzed in 112 of the 184 PRL cases, and the count was higher than 2×10⁶/ml.

Control subjects were eligible for study participation if they fulfilled the following criteria: age >18 years at first pregnancy, at least 1 pregnancy to term, no chronic pathology, no personal or family history of thrombosis, no history of obstetric complications (miscarriage or fetal death, pre-eclampsia, eclampsia, intrauterine growth restriction, placental abruption), and not taking concomitant anticoagulant or anti-aggregation therapies during pregnancy.

The genetic analysis entailed the collection of a saliva sample (by oral mucosal smear) or blood sample, DNA extraction (by digestion and selective precipitation with ethanol), and genotyping of the prothrombotic genetic variables that were identified as (gene-rs) using the standard FVL-PT panel and Thrombo inCode® (in the following also “TiC”, Ferrer inCode, Barcelona, Spain) (Soria et al., 2014). The FVL-PT panel consisted of the F5-rs6025 and F2-rsl799963 genetic variants. The TiC panel included 12 genetic variables: F2-rs1799963, F5-rs6025, F12-rs1801020, F13-r55985, AB0-rs8176719, rs7853989, rs8176743, and rs8176750 (all 4 AB0 rs forming the haplotype for identification of A1 AB0 group carriers). The genetic analysis was performed at Gendiag.exe.

Study Variables and Data Analysis

The clinical variables that we considered were age, family history of VTE, and week at which the pregnancy loss occurred. All variables were analyzed for patients with recurrent miscarriage and controls.

The association between genetic variables and recurrent miscarriages was determined, taking into account the confounding effect of age. For this purpose, a logistic regression model was fitted, including the individual genetic variable and age as the independent variables in the model.

For the development of the Thrombo InCode for repeated pregnancy loss (TiC-RPL) risk score, age and genetic variables that were individually associated with recurrent miscarriage (p<0.10) were analyzed by multivariate logistic regression. Hosmer-Lemeshow test was used to assess the correct calibration of the models. TiC-RPL score was compared against FVL+PT, a binary score that was defined as 1 in the presence of the F5-rs6025 or F2-rs17799963 risk allele and 0 otherwise.

The predictive capacity of the risk scores was evaluated using the area under the receiver operating characteristic curve (AUC; larger values indicate better discrimination) (Hanley and Hajian-Tilaki, 1997). DeLong test was used to compare AUC values between the 2 scores. Standard measures of sensitivity, specificity, positive and negative predictive values (PPV, NPV), and positive and negative likelihood ratios (PLR, NLR) (Attia, 2003) were calculated. These measures were compared between scores using the R package DTComPair (http://CRAN.R-project.org/package=DTComPair), which implements several methods for each of the measures. Briefly, sensitivity and specificity were compared by McNemar test, PPV and NPV were compared using a generalized score statistic (Leisenring, Alonzo and Pepe, 2000), and likelihood ratios were compared using a regression model approach (Gu and Pepe, 2009).

The cut-off for high risk using the FVL-PT score was 0.5 (which is equivalent to define as high risk individuals with the presence of any risk allele), and for the TiC-RPL score, this threshold was the point on the ROC curve that corresponded to a sensitivity of approximately 70%. The cut-off for relevant thrombophilia that could be responsible for RPL was established as the presence of any thrombophilia for which the risk was similar or higher to that for F5-rs6025.

Cross-validated AUC was also computed to correct for any overoptimism bias, because all samples were used to fit the regression model for the TiC-RPL score. For this purpose, a leave-one-out cross-validation (LOOCV) approach was used. Briefly, 1 sample was eliminated, and a new regression model was fitted with the remaining samples to estimate their coefficients. The predicted risk for the omitted sample was then computed, based on the new model. This step was repeated until every sample was left out once. The newly generated risk values were used to calculate the corrected AUC.

All calculations were performed using R, version 3.1.3 (R Development Core Team, 2015).

### RESULTS

### Patient Characteristics

Of the 364 subjects who participated in this study, there were 180 healthy women in the control group and 184 in the RPL group. Age differed (p<0.001) between the healthy control and RPL groups (median 31 vs 35 years, respectively). Among the genetic variables, F12-rs1801020 (p=0.019), F13-rs5985 (p=0.062), F2-rs1799963 (p=0.037), and AB0 haplotype (p=0.030) were individually associated with RPL (Table 3), as can be seen herein below:

TABLE 3
Prevalence of genetic variables in patients with recurrent miscarriage

| | Control | | Recurrent miscarriage | | | P value** |
|---|---|---|---|---|---|---|
| | (n = 183) | | (n = 184) | | P value* | (age-adjusted) |
| F12-rs1801020 | | | | | 0.202 | 0.019 |
| 0 | 121 | (67.2%) | 107 | (58.2%) | | |
| 1 | 48 | (26.7%) | 63 | (34.2%) | | |
| 2 | 11 | (6.11%) | 14 | (7.61%) | | |
| SERPINA10-rs2232698 | | | | | 0.751 | 0.533 |
| 0 | 176 | (97.8%) | 178 | (96.7%) | | |
| 1 | 4 | (2.22%) | 6 | (3.26%) | | |
| SERPINC1-rs121909548 | | | | | 0.244 | 0.981 |
| 0 | 178 | (98.9%) | 184 | (100%) | | |
| 1 | 2 | (1.11%) | 0 | (0.00%) | | |
| F5-rs6025 | | | | | 0.565 | 0.633 |
| 0 | 176 | (97.8%) | 177 | (96.2%) | | |
| 1 | 4 | (2.22%) | 7 | (3.80%) | | |
| F5-rs118203906 | | | | | . | . |
| 0 | 180 | (100%) | 184 | (100%) | | |
| F5-rs118203905 | | | | | . | . |
| 0 | 180 | (100%) | 184 | (100%) | | |
| F13-rs5985 | | | | | 0.394 | 0.062 |
| 0 | 109 | (60.6%) | 99 | (53.8%) | | |
| 1 | 61 | (33.9%) | 71 | (38.6%) | | |
| 2 | 10 | (5.56%) | 14 | (7.61%) | | |
| F2-rs1799963 | | | | | 0.006 | 0.037 |
| 0 | 178 | (98.9%) | 170 | (92.4%) | | |
| 1 | 2 | (1.11%) | 14 | (7.61%) | | |
| ABO | | | | | 0.163 | 0.030 |
| 0 | 106 | (58.9%) | 126 | (68.5%) | | |
| 1 | 62 | (34.4%) | 49 | (26.6%) | | |
| 2 | 12 | (6.67%) | 9 | (4.89%) | | |

0-2, number of minor alleles
Data are expressed as n (%)
*p-value for standard chi-square test
**p-value adjusted for the confounding effect of age

As can be seen from this table, four further genetic variants, which were included in this study and are part of the TiC panel, are quite surprisingly not individually associated with miscarriage/RPL risk.

### Development of the TiC-RPL Risk Model

Table 4 shows the genetic and clinical variables that were included in the TiC-RPL risk score. F5-rs6025 was incorporated, based on a meta-analysis (Skeith et al., 2016; Sergi et al., 2014, Rey et al., 2003). The weights that were assigned to each variable were defined from a meta-analysis for F5-rs6025 and F2-rs1799963 (Rey et al., 2003) and by multivariate logistic regression for the rest of the variables. These genetic variable (and additionally preferably age) are the TiC-RPL combination which constitutes the present invention.

TABLE 4
Odds ratios for age, smoking, and genetic variables
in patients with recurrent miscarriage

| Variable | OR (95% CI) | P value |
|---|---|---|
| Age | 1.24 (1.17-1.32) | <0.0001 |
| F12 | 1.67 (1.14-2.47) | 0.0097 |
| F13 | 1.49 (1.02-2.20) | 0.0401 |
| A1 | 1.89 (1.17-3.09) | 0.0103 |
| F2 | 2.32* | |
| F5 | 2.01* | |

F12, F12-rs1801020;
F13, F13-rs5985;
F2, F2-rs1799963;
A1: AB0-rs8176719-rs7853989-rs8176743-rs8176750;
F5, F5-rs6025
*OR extracted from meta-analysis
Data are expressed as OR (95% CI).

For the TiC-RPL score to identify patients who are at risk, a cut-off that yielded a sensitivity of 70.65% and specificity of 67.78% was selected. By comparison, for the FVL-PT to identify such patients at risk, the presence of any risk allele in F5-rs6025 and F2-rs1799963 was selected as cut-off that yielded a sensitivity of 11.4% and specificity of 96.7%.

### Accuracy and Validation of the Risk Model

The TiC-RPL score had an area under the ROC curve of 0.763 (0.715-0.811), a sensitivity of 70.65%, and a specificity of 67.78%. It had a PPV of 69.15%, an NPV of 69.32%, a PLR of 2.19, an NLR of 0.43 (Table 5), and a cross-validated AUC value of 0.742 (0.682-0.784). The FVL-PT score did not distinguish between patients who did and did not experience an RPL as well (0.763 vs 0.540; p<0.0001, see also FIG. 2). The sensitivity of the TiC-RPL score was significantly higher than that of the FVL-PT (70.65% vs. 11.4%; p<0.0001), whereas its specificity was lower (67.78% vs. 96.7%; p<0.0001). The NPV of the TiC-RPL score exceeded that of the FVL-PT score (69.32% vs. 51.63%; p<0.0001), but its PPV scores were similar (69.15% vs. 77.8%; p=0.2772). The NLR of the TiC-RPL score was also significantly better versus the FVL-PT, but their PLRs were similar (Table 5).

TABLE 5
TiC panel performance metrics and comparison with standard
FVL-PT panel in patients with recurrent miscarriage

| Variable | TiC | FVL-PT | P value |
|---|---|---|---|
| AUC (95% CI) | 0.763 (0.715-0.811) | 0.540 (0.514-0.567) | <0.0001 |
| (p-value) | (<0.0001) | (0.003) | |
| Sensitivity | 70.65% | 11.4% | <0.0001 |
| Specificity | 67.78% | 96.7% | <0.0001 |
| PPV | 69.15% | 77.8% | 0.2772 |
| NPV | 69.32% | 51.6% | <0.0001 |
| PLR | 2.19 | 3.42 | 0.3218 |
| NLR | 0.43 | 0.92 | <0.0001 |
| Calibration (p) | 0.616 | >0.999 | |

AUC, area under the curve (measure of discrimination capability);
PPV, positive predictive value;
NPV, negative predictive value;
PLR, positive likelihood ratio;
NLR, negative likelihood ratio;
FVL-PT, F5-rs6025 + F2-rs1799963

Data are expressed as OR (95% IC) or %

The proportion of RPL patients who were classified as high- or low-risk according to the FVL-PT or TiC-RPL score was also compared. Most patients who suffered an RPL (88.59%) were identified by the FVL-PT score as low-risk. Notably, among these patients, 68.1% was reclassified as high-risk by the TiC-RPL score! TiC-RPL considered 70.65% of patients who suffered an RPL to be at high risk of developing RPL.

TiC-RPL identified 130 (70.65%) of the 184 RPL women as being at high risk for RPL. All patients at high risk for PL/RPL according to TiC-RPL could be considered patients in whom thrombo-prophylaxis could be suggested.

### Discussion

The TiC-RPL score that has been developed in this study identifies women in whom RPL is associated with significant thrombophilia. This identification can guide personalized approach to prevent the development of miscarriage or RPL events.

By multivariate analysis, a model with 8 genetic variants (and preferably also including age) was developed, which defined the algorithm for the TiC-RPL, which initially allowed the patients to be classified as being at high or low risk of RPL. Among patients in the TiC-RPL-based high-risk group, 69.15% eventually suffered an RPL, whereas 30.68% of the low-risk group did so (Table 5). By comparison, 77.78% of high-risk patients according to FVL-PT score experienced an RPL. Similarly, 48.36% of patients in the low-risk group, based on FVL-PT score, did so. Nevertheless, clearly TiC-RPL detects more women with RPL as being high-risk (130 vs 21 who were identified by FVL-PT); yet, TiC-RPL classifies fewer women with RPL as low-risk (54 vs 163 as classified by FVL-PT).

The contribution of thrombophilia to pregnancy loss and other adverse outcomes in pregnancy remains debated (Battinelli et al., 2013). Thus, the guidelines of the American College of Chest Physicians recommend against screening for inherited thrombophilia in women with a history of pregnancy complications (Bates et al., 2012). These conflicting results on thrombophilia are most likely attributed to the use of a single-marker marginal analysis approach using F5-rs6025 alone or in combination with F2-rs1799963. This standard approach might suffer from low power and poor reproducibility. One useful strategy for solving these problems is marker set analysis, in which a combination of genetic markers is determined and evaluated regarding its predictive power.

As a result, a new algorithm (see above) has been developed from clinical and genetic markers. Firstly, the present study shows, that age was significantly associated with RPL—women with RPL were older than those with non-complicated pregnancies (35 versus 31 years, p<0.001), and are used as a control.

Because the present study was focused on idiopathic RPL, patients with clinical factors, such as obesity, that have been linked to RPL and that clinicians should consider in evaluating patients with RPL (Smith ML and Schust DJ, 2011), have been excluded.

The genetic variants in the present algorithm have been individually linked to RPL. The association of F2-rs1799963 and F5-rs6025 with RPL has been studied extensively (Simcox et al., 2015; Skeith et al., 2016; Sergi et al., 2014; Rey et al., 2013; Rodger et al., 2010; Lissalde-Lavigne et al., 2005; Kovalevsky et al., 2004), although the clinical sensitivity has not been reported (Bradley et al., 2012); clinical sensitivity however has been shown to be low in the present study. These two genetic variants are currently used as a standard panel for the determination of the presently described risk.

The exact mechanism by which inherited thrombophilia causes RPL is unknown. It has been suggested that inherited thrombophilia impairs placental function by causing arterial or venous thrombosis at the maternal-foetal interface. Also, thrombophilia has been proposed to effect syncytio-trophoblast invasion of the maternal blood vessels, leading to the formation of micro-thrombosis at the site of implantation and thus resulting in RPL (Abu-Heija, 2014).

The avoid any bias on the basis of the selected patient population, the F5-rs6025 has been included based on the literature and the weights for F5-rs6025 and F2-rs1799963 have been taken from a published meta-analyses with 3753 women (Rey et al., 2003).

One of the major achievements of this study is the development of a particular successful combination of genetic variables, preferably in combination with the variable “age” as well as the development of an algorithm through marker-set analysis, in which a set of genetic markers is assembled. This combination generates better results than F5-rs6025 and F2-rs1799963 genetic variants. A further advantage provided herein is the selection of the variables and the analysis that was performed to characterize the goodness of the 2 algorithms: TiC-RPL and FVL-PT (Greenland et al., 2008; Attia, 2003). Most studies limit this analysis to the association between the marker and RPL. F5-rs6025 might have a strong association with RPL (OR: 2.01) (Rey et al., 2003) with a good PPV (herein, it is 77.78%, combined with F2-rs1799963), but these variants are uncommon in RPL women, limiting their clinical value (in our case, the sensitivity was 11.41%). The use of only 2 variants with low sensitivity, such as F5-rs6025 and F2-rs1799963, might explain the lack of reproducible results with these variants in identifying people at risk and selecting patients for thrombo-prophylaxis (the AUC for FVL-PT was also low, AUC=0.540).

With the present study, the clinician is provided with an algorithm that identifies women who are at risk of experiencing a miscarriage or developing RPL. This combination/algorithm could be used to predict the possibility for a pregnancy loss in general, or after the first (or any further) pregnancy loss to identify such women. It could also be applied to women with confirmed RPL to identify those who are at high risk of RPL in whom thrombo-prophylaxis might be indicated. To recommend thrombo-prophylaxis, women who are at high risk for RPL have been identified in whom the presence of thrombophilia (as a single genetic variant or a combination, according to the proposed multivariate model) is associated with RPL to a degree that is similar to or stronger than F5-rs6025 (OR 2.01) which is the threshold that is used by most guidelines as the level of thrombophilia that requires intervention. Applying this criterion, of 130 high-risk women in the RPL group, 91 (70%) could be considered patients in whom thrombophilia is relevant and thrombo-prophylaxis can be suggested. Using this criterion in an ongoing pilot study, of 80 women who were at high risk for RPL, 37 had significant thrombophilia (as a single genetic variant or a combination, according to the proposed multivariate model) to extent that was similar to or stronger than F5-rs6025. All 37 were treated with prophylactic doses of LMWH, and 33 of them (89.2%) experienced a pregnancy to term.

In summary, this application provides for a clinical-genetic risk score that is significantly better than FVL-PT, as demonstrated by its greater AUC value, sensitivity, negative likelihood ratios, and sensitivity (70.7%) in identifying RPL women. The recommendation of thrombo-prophylaxis might be appropriate for those with significant thrombophilia—similar to or stronger than FVL. The use of the present clinic-genetic risk scores, is useful in solving the contradictory results regarding inherited thrombophilia in RPL. Patients who are identified as being at high risk by the TiC-RPL risk score and with significant thrombophilia are likely to benefit from thrombo-prophylaxis. A highly sensitive predictive tool, such as the TiC-RPL score, is now available to improve the infertility that is associated with thrombophilia, considering the low risk of possible thrombo-prophylactic measures.

Sequence Information Relating to SNPs

| | |
|---|---|
| rs1801020 | SEQ ID NO: 1 |
| rs5985 | SEQ ID NO: 2 |
| rs1799963 | SEQ ID NO: 3 |
| rs6025 | SEQ ID NO: 4 |
| rs8176719 | SEQ ID NOs: 5 and 6 |
| rs7853989 | SEQ ID NO: 7 |
| rs8176743 | SEQ ID NO: 8 |
| rs8176750 | SEQ ID NOs: 9 and 10 |
| rs2232698 | SEQ ID NO: 11 |
| rs121909548 | SEQ ID NO: 12 |
| rs118203906 | SEQ ID NO: 13 |
| rs118203905 | SEQ ID NO: 14 |

### REFERENCES

Abu-Heija A. Thrombophilia and Recurrent Pregnancy Loss: Is heparin still the drug of choice? Sultan Qaboos Univ Med J. 2014; 14(1): e26-e36.
Andersen A M N, Wohfahrt J, Christens P, Olsen J, Melbye M. Maternal age and fetal loss: population based register linkage study. BMJ 2000;329:1708-1712.
Ariëns R A, Philippou H, Nagaswami C, Weisel J W, Lane D A, Grant P J. The factor XIII V34L polymorphism accelerates thrombin activation of factor XIII and affects cross-linked fibrin structure. Blood. 2000; 96(3): 988-995.
Attia J. Moving beyond sensitivity and specificity: using likelihood ratios to help interpret diagnostic tests. Aust Prescr. 2003; 26(5): 111-113.
Bates S M, Greer I A, Middeldorp S, Veenstra D L, Prabulos A M, Vandvik P O. VTE, thrombophilia, antithrombotic therapy, and pregnancy: Antithrombotic Therapy and Prevention of Thrombosis, 9th ed: American College of Chest Physicians Evidence-Based Clinical Practice Guidelines. Chest. 2012; 141(2 Suppl): e691S-e736S.
Battinelli E M, Marshall A, Connors J M. The Role of Thrombophilia in Pregnancy. Thrombosis. 2013; 2013: 516420.
Bradley L A, Palomaki G E, Bienstock J, Varga E, Scott J A. Can Factor V Leiden and prothrombin G20210A testing in women with recurrent pregnancy loss result in improved pregnancy outcomes?: Results from a targeted evidence-based review. Genet Med. 2012; 14(1): 39-50.
Cao Y, Zhang Z, Xu J, Yuan W, Wang J, Huang X, Shen Y, Du J. The association of idiopathic recurrent pregnancy loss with polymorphisms in hemostasis-related genes. Gene. 2013; 530(2): 248-252.
Cohn D M, Goddijn M, Middeldorp S, Korevaar J C, Dawood F, Farquharson R G. Recurrent miscarriage and antiphospholipid antibodies: Prognosis of subsequent pregnancy. J Thromb Haemost. 2010; 8(10): 2208-2213.
Dossenbach-Glaninger A, van Trotsenburg M, Dossenbach M, Oberkanins C, Moritz A, Krugluger W, Huber J, Hopmeier P. Plasminogen activator inhibitor 1 4G/5G polymorphism and coagulation factor XIII Val34Leu polymorphism: impaired fibrinolysis and early pregnancy loss. Clin Chem. 2003; 49(7): 1081-1086.
Dossenbach-Glaninger A, van Trotsenburg M, Krugluger W, Dossenbach M R, Oberkanins C, Huber J, Hopmeier P. Elevated coagulation factor VIII and the risk for recurrent early pregnancy loss. Thromb Haemost. 2004; 91(4): 694-699.
Dossenbach-Glaninger A, van Trotsenburg M, Oberkanins C, Atamaniuk J. Risk for early pregnancy loss by factor XIII Val34Leu: the impact of fibrinogen concentration. J Clin Lab Anal. 2013; 27(6): 444-449.
George L, Granath F, Johansson A L V, Olander B, Cnattingius S. Risks of repeated miscarriage. Paedriatic Perinat Epidemiol 2006;20:119-126
Greenland P. Comments on “Evaluating the added predictive ability of a new marker: From area under the ROC curve to reclassification and beyond” by M. J. Pencina, R. B. D' Agostino Sr, R. B. D' Agostino Jr, R. S. Vasan, Stat Med. 2008; 27(2): 188-190.
Gu W, Pepe MS. Estimating the capacity for improvement in risk prediction with a marker. Biostatistics. 2009 Jan ;10(1):172-86
Hanley J A, Hajian-Tilaki K O. Sampling variability of nonparametric estimates of the areas under receiver operating characteristic curves: an update. Acad Radiol 1997; 4(1): 49-58.
Kovalevsky G, Gracia C R, Berlin J A, Sammel M D, Barnhart K T. Evaluation of the association between hereditary thrombophilias and recurrent pregnancy loss: a meta-analysis. Arch Intern Med. 2004; 164(5): 558-563.
Leisenring W, Alonzo T, Pepe M S. Comparisons of predictive values of binary medical diagnostic tests for paired designs. Biometrics. 2000 Jun;56(2):345-51
Li J, Wu H, Chen Y, Wu H, Xu H, Li L. Genetic association between FXIII and β-fibrinogen genes and women with recurrent spontaneous abortion: a meta-analysis. J Assist Reprod Genet. 2015; 32(5): 817-825.
Lim B C, Ariëns R A, Carter A M, Weisel J W, Grant P J. Genetic regulation of fibrin structure and function: complex gene-environment interactions may modulate vascular risk. Lancet. 2003; 361(9367): 1424-1431.
Lissalde-Lavigne G, Fabbro-Peray P, Cochery-Nouvellon E, Mercier E, Ripart-Neveu S, Balducchi J P, Daurès J P, Perneqer T, Quéré I, Dauzat M, et al. Factor V Leiden and prothrombin G20210A polymorphisms as risk factors for miscarriage during a first intended pregnancy: the matched case-control ‘NOHA first’ study. J Thromb Haemost. 2005; 3(10): 2178-2184.
Mannucci P M, Franchini M. The real value of thrombophilia markers in identifying patients at high risk of venous thromboembolism. Expert Rev Hematol. 2014; 7(6): 757-765.
Marietta M, Facchinetti F, Sgarbi L, Simoni L, Bertesi M, Torelli G, Volpe A. Elevated plasma levels of factor VIII in women with early recurrent miscarriage. J Thromb Haemost. 2003; 1: 2536-2539.
Martinelli I, Bucciarelli P, Mannucci P M. Thrombotic risk factors: basic pathophysiology. Crit Care Med. 2010; 38(2 Suppl): S3-9.
Middeldorp S. Anticoagulation in pregnancy complications. Hematology Am Soc Hematol Educ Program. 2014; 2014(1): 393-399.
Ogasawara M S, Aoki K, Katano K, Ozaki Y, Suzumori K. Factor XII but not protein C, protein S, antithrombin III, or factor XIII is a predictor of recurrent miscarriage. Fertil Steril. 2001; 75(5): 916-919.
Practice Committee of American Society for Reproductive Medicine. Definitions of infertility and recurrent pregnancy loss: a committee opinion. Fertil Steril. 2013; 99(1): 63.
R Development Core Team (2015). R: A Language and Environment for Statistical Computing. R Foundation for Statistical Computing, Vienna, Austria. URL://www.R-project.org/.
Rai R, Regan L. Recurrent miscarriage. Lancet 2006;368:601-611.
Rey E, Kahn S R, David M, Shrier I. Thrombophilic disorders and fetal loss: a meta-analysis. Lancet. 2003; 361(9361): 901-908.
Rodger M A, Betancourt M T, Clark P, Lindqvist P G, Dizon-Townson D, Said J, Seliqsohn U, Carrier M, Salomon O, Greer I A. The association of factor V leiden and prothrombin gene mutation and placenta-mediated pregnancy complications: A systematic review and meta-analysis of prospective cohort studies. PLoS Med. 2010; 7(6): e1000292.
Sergi C, Al Jishi T, Walker M. Factor V Leiden mutation in women with early recurrent pregnancy loss: a meta-analysis and systematic review of the causal association. Arch Gynecol Obstet. 2014; 291(3): 671-679.
Simcox LE, Ormesher L, Tower C, Greer I A. Thrombophilia and Pregnancy Complications. Int J Mol Sci. 2015; 16(12): 28418-28428.
Skeith L, Carrier M, Kaaja R, Martinelli I, Petroff D, Schleussner E, Laskin CA, Rodger M A. A meta-analysis of low-molecular-weight heparin to prevent pregnancy loss in women with inherited thrombophilia. Blood. 2016; 127(13): 1650-1655.
Smith M L and Schust D J. Endocrinology and recurrent early pregnancy loss. Semi Reprod Med 2011; 29: 482-490.
Song J, Chen F, Campos M, Bolgiano D, Houck K, Chambless L E, Wu K K, Folsom A R, Couper D, Boerwinkle E, et al. Quantitative Influence of ABO Blood Groups on Factor VIII and Its Ratio to von Willebrand Factor, Novel Observations from an ARIC Study of 11,673 Subjects. PLoS One. 2015; 10(8): e0132626.
Soria J M, Morange P-E, Vila J, Souto J C, Moyano M, Trégouët D A, Mateo J, Saut N, Salas E, Elosua R. Multilocus genetic risk scores for venous thromboembolism risk assessment. J Am Heart Assoc. 2014; 3(5): e001060.
Wells P S, Anderson J L, Scarvelis D K, Doucette S P, Gagnon F. Factor XIII Val34Leu variant is protective against venous thromboembolism: A HuGE review and meta-analysis. Am J Epidemiol. 2006; 164(2): 101-109.
Ziakas P D, Poulou L S, Pavlou M, Zintzaras E. Thrombophilia and venous thromboembolism in pregnancy: A meta-analysis of genetic risk. Eur J Obstet Gynecol Reprod Biol. 2015; 191: 106-111.

1. A method for the risk assessment in a human female subject for experiencing a miscarriage and/or recurrent pregnancy loss, comprising the steps of determining in a sample isolated from said subject the presence of factor XII C46T (rs1801020), factor XIII Val34Leu (rs5985), Factor II (prothrombin) G20210A (rs1799963), factor V Leiden Arg506Gln (rs6025), ABO haplotype (consisting of ABO rs8176719, ABO rs7853989, ABO rs8176743, and ABO rs8176750), or respective SNPs in strong linkage disequilibrium with these variants, whereby the presence of these genetic variables (or the absence of any A1 allele in the case of ABO gene) is indicative of a risk of experiencing a miscarriage and/or a recurrent pregnancy loss (RPL).

2. A method for the diagnosis of a risk for experiencing a miscarriage and/or recurrent pregnancy loss in a human female subject comprising the steps of determining in a sample isolated from said subject the presence of factor XII C46T (rs1801020), factor XIII Val34Leu (rs5985), Factor II (prothrombin) G20210A (rs1799963), factor V Leiden Arg506Gln (rs6025), ABO haplotype (consisting of ABO rs8176719, ABO rs7853989, ABO rs8176743, and ABO rs8176750), or respective SNPs in strong linkage disequilibrium with these variants, whereby the presence of these genetic variables (or the absence of any Al allele in the case of ABO gene) is indicative of a risk of experiencing a miscarriage and/or a recurrent pregnancy loss (RPL).

3. The method as defined in claim 1 wherein the method is to determine or diagnose the risk of experiencing an RPL in a subject who has already experienced at least one miscarriage.

4. The method as defined in claim 1 further comprising determining the age of the subject.

5. The method according to claim 1 wherein the sample is an oral tissue sample, scraping, or wash or a biological fluid sample, preferably saliva, urine or blood.

6. The method according to claim 1 wherein the presence or absence of the polynucleotide is identified by amplifying or failing to amplify an amplification product from the sample, wherein the amplification product is preferably digested with a restriction enzyme before analysis and/or wherein the SNP is identified by hybridizing the nucleic acid sample with a primer label which is a detectable moiety.

7. The method according to claim 1 wherein the presence or absence of the polynucleotide is identified by hybridization to specific Hairloop™ probes spotted on a microarray, by allele-specific PCR, by KASP genotyping chemistry or TaqMan Assays.

8. A method of determining the probability of an human female subject of presenting a miscarriage and/or RPL based on the presence of 1 to P classical risk factors and 1 to J polymorphisms selected from the group of factor XII C46T (rs1801020), factor XIII Val34Leu (rs5985), Factor II (prothrombin) G20210A (rs1799963), factor V Leiden Arg506Gln (rs6025), ABO rs8176719, ABO rs7853989, ABO rs8176743, and ABO rs8176750, or respective SNPs in strong linkage disequilibrium with these variants, using the formula:
Estimating the Risk of (Repeated) Pregnancy Loss.
The individual estimation of the risk of (repeated) pregnancy loss is based on a logistic regression model. The aim of this model is to calculate the probability that a person has of presenting (repeated) pregnancy loss according to his/her genetic, sociodemographic and clinical characteristics. To calculate this probability we use the following equation:
Probability (Y=1|X₁, . . . , Xₙ)=1/1+exp(β₀+β₁x₁+ . . . +βₙxₙ₊β_{f·g}x_{f}·x_{g}+ . . . +β_{h·i}xₕ·xᵢ),
Probability (Y=1|X₁, . . . , Xₙ)=1/1+exp(β₀+β₁x₁+ . . . +βₙxₙ₊β_{f·g}x_{f}·x_{g}+ . . . +β_{h·i}xₕ·xᵢ),
Probability (Y=1|X₁, . . . , Xₙ)=1/1+exp(β₀+β₁X₁+ . . . +βₙXₙ) Function 1
wherein:
Probability (Y=1|x₁, . . . , xₙ)=probability of presenting a pregnancy loss or a repeated pregnancy loss associated to thrombophilia in a particular individual with concrete and measurable characteristics in a number of variables 1, . . . , n. This probability could range between 0 and 1;
Exp=exponential natural base;
β₀=coefficient that defines the risk (the probability) of a pregnancy loss or a repeated pregnancy loss associated to thrombophilia non related with the variables 1 to n. This coefficient can take a value from −∞ to +∞ and is calculated as the natural logarithm of the incidence of venous thrombosis
Probability (Y=1|X₁, . . . , Xₙ)=1/1+exp (β₀+β₁x₁+ . . . +βₙxₙ₊β_{f·g}xᵣ·x_{g}+ . . . +β_{h·i}xₕ·xᵢ),
in the population;
β₁=regression coefficient that expresses the risk (higher or lower) to present a pregnancy loss or a repeated pregnancy loss associated to thrombophilia associated with the value/presence of the predictor variable x₁. This coefficient can take a value from − [symbol] to + [symbol] ;
x₁=value taken by the predictor variable x1 in an individual. The range of possible values depends on the variable;
βₙ=regression coefficient that expresses the risk (higher or lower) to present thrombosis associated with the value/presence of the predictor variable xₙ. This coefficient can take a value from −∞ to +∞;
xₙ=value taken by the predictor variable xₙ in an individual. The range of possible values depends on the variable.

9. The method according to claim 1, wherein no further genetic variables are determined.

10. A computer program or a computer-readable media containing means for carrying out a method as defined in claim 1.

11. A kit comprising reagents for detecting the identity of the nucleotide selected from the group of factor XII C46T (rs1801020), factor XIII Val34Leu (rs5985), Factor II (prothrombin) G20210A (rs1799963), factor V Leiden Arg506Gln (rs6025), ABO (rs8176719, rs7853989, rs8176743, and rs8176750), or respective SNPs in strong linkage disequilibrium with these variants, and instructions for use.

12. The kit as defined in claim 11 which comprises one or more primer pairs specific for the amplification of a region comprising factor XII C46T (rs1801020), factor XIII Val34Leu (rs5985), Factor II (prothrombin) G20210A (rs1799963), factor V Leiden Arg506Gln (rs6025), ABO blood group rs8176719, ABO (rs7853989, rs8176743, and rs8176750), or for respective SNPs in strong linkage disequilibrium with these variants.

13. The kit according to claim 12, which consists of the primer pairs of claim 12, the instructions for use, and reagents suitable for end-point, fluorescence-PCR chemistry.

14. The kit according to claim 13, wherein said reagents are dual hydrolysis probes, MgCl₂ and DNA polymerase.

15. The kit according to claim 13, wherein the primer pairs are

| Detected Variant | Primer | Sequence (5′→3′) |
|---|---|---|
| rs1799963 | 9963-F | TTGTGTTTCTAAAACTATGGTTCC |
| | 9963-R | AGTAGTATTACTGGCTCTTCCT |
| rs6025 | 6025-F | TCTGAAAGGTTACTTCAAGGAC |
| | 6025-R | ATCGCCTCTGGGCTAATA |
| rs1801020 | 1020-F | TGATCTGGACTCCTGGATAG |
| | 1020-R | ATCCTGGTTCCCACAGCAC |
| rs5985 | 5985-F | TCCACCCAATAACTCTAATGC |
| | 5985-R | GTATGCTCATACCTTGCAGG |
| rs7853989 | 3989-F | ATCCACCTCGCTGAGGAAG |
| | 3989-R | CCACCGTGTCCACTACTATG |
| rs8176719 | 6719-F | TCTCCATGTGCAGTAGGAAG |
| | 6719-R | CAATGGTGGTGTTCTGGAG |
| rs8176743 | 6743-F | CAGCGAGGTGGATTACCTG |
| | 6743-R | CCGGCGCTCGTAGGTGAA |
| rs8176750 | 6750-F | GCTGAGGTTCACTGCGGTG |
| | 6750-R | TTACTCACAACAGGACGGAC |

16. The method as defined in claim 2 wherein the method is to determine or diagnose the risk of experiencing an RPL in a subject who has already experienced at least one miscarriage.

17. The method as defined in claim 2 further comprising determining the age of the subject.

18. The method as defined in claim 3 further comprising determining the age of the subject.
